(19) United States
(12) Patent Application Publication
(10) Pub. No.: US 2024/0285433 A1
(43) Pub. Date: Aug. 29, 2024

(54) GLAUCOMA DRAINAGE DEVICE WITH EXTENSIBLE TUBE AND LOCKING TIP

(71) Applicant: Nicolas Hamouche, Ames, IA (US)

(72) Inventor: Nicolas Hamouche, Ames, IA (US)

(21) Appl. No.: 18/590,359

(22) Filed: Feb. 28, 2024

Related U.S. Application Data
(60) Provisional application No. 63/448,888, filed on Feb. 28, 2023.

(51) Int. Cl. A61F 9/007

(52) U.S. Cl. CPC A61F 9/00781; A61F 2220/0075; A61F 2250/0081

(57) ABSTRACT

Methods and devices for draining intraocular fluid from an eye and treating glaucoma in humans or animals are provided. In some embodiments, the device has a scleral plate with a groove and/or locking mechanism and an extensible tube or tubes having at least one lumen. In some embodiments, the distal portion of the extensible tube has a terminal open end adapted to be placed in an intraocular chamber of an eye and configured to prevent extrusion of the terminal open end from the intraocular chamber following placement.

### CROSS REFERENCE TO RELATED APPLICATIONS

This application claims priority under 35 U.S.C. § 119 to provisional patent application U.S. Ser. No. 63/448,888 filed Feb. 28, 2023. The provisional patent application is herein incorporated by reference in its entirety, including without limitation, the specification, claims, and abstract, as well as any figures, tables, appendices, or drawings thereof.

### TECHNICAL FIELD

The present disclosure relates generally to an improved glaucoma drainage implant with extendable tube and locking tip and associated methods of use.

### BACKGROUND

Glaucoma drainage implants (GDIs) are commonly used for lowering the intraocular pressure (IOP) in the treatment of glaucoma. They are typically used when other surgeries such as minimally invasive glaucoma surgery (MIGS) or trabeculectomy have failed. More recently, drainage implants are being considered as earlier options in the management of glaucoma. In 2017, around 20,000 such implants were used in the United States. During a GDI surgery, a plastic end-plate (silicone or polypropylene) is sutured to the sclera 8-10 mm posterior to the corneal limbus. A silicone tube, ranging in size from 0.3 mm to 0.64 mm outer diameter and 0.127 mm to 0.3 mm inner diameter, connected to the plate is then cut to an adequate length and inserted into the anterior chamber (AC) of the eye through a limbal fistula usually created by a 23 G needle. The tube is cut so that it extends for 1-3 mm into the AC. Effort is made to keep the tube as distant from the corneal endothelium as possible. Alternatively, the tube can be inserted into the posterior chamber or the vitreous cavity.

Like any other glaucoma surgical intervention, current GDIs have several complications, including early hypotony, early hypertensive phase, and long term corneal endothelial loss leading to corneal failure necessitating corneal transplant surgery.

Early hypotony (immediately after or within a few weeks from surgery) is one of the biggest challenges of glaucoma surgery, including GDI surgery, and can result in significant visual loss in around 20% of patients. Besides low IOP, patients can have shallow or flat AC, hypotony maculopathy, choroidal detachment, or even choroidal hemorrhage. In non-restricted GDIs such as Moltenos or Baerveldts, early hypotony is common since the bleb resistance around the plate remains low for around 6 weeks after surgery. To prevent early hypotony, some devices, such as the Ahmed tube, come with a “valve” mechanism that is supposed to restrict flow when the IOP is less than 8-10 mmHg. However, such a mechanism is not always reliable, particularly in the days immediately following implant when the valve tends not to close after initial perfusion. Hypotony after a valved tube insertion is still a possibility in up to 13% of cases. When a non-valved GDI is used, the tube is either ligated with an absorbable suture and/or otherwise occluded for several weeks after surgery. During that time, a fibrous capsule forms around the scleral plate and the risk of hypotony is reduced. When non-absorbable sutures are used to ligate or occlude the tube, a separate procedure is required to reverse the obstruction. Besides the hypotony resulting from the low resistance of the bleb around the plate, all implants can result in immediate postoperative hypotony because of aqueous leakage around the tube due to tissue distension and manipulation while creating the limbal fistula and inserting the tube through it. Thus, there remains a need in the art for an eye drainage device with a decreased risk of immediate/early hypotony.

An additional complication of current GDIs is early hypertensive phase. The early hypertensive phase after a GDI can last for several months and is due to transient low capsule permeability around the plate. It is thought to be due to the early flow of pro-inflammatory aqueous immediately after surgery. Hence, hypertension is more common after unrestricted, “early flow” devices such as the Ahmed valve (40-80%), compared to the “early restricted” devices such Baerveldt and Molteno (20-30%) where flow of aqueous is delayed for several weeks after surgery. Thus, there remains a need in the art for an eye drainage device with a decreased risk of early hypertensive phase.

A further complication of current GDIs is endothelial cell loss. The corneal endothelium is a monolayer of hexagonal cells lining the posterior surface of the cornea that regulates corneal hydration. These cells don't regenerate, and their damage or loss leads to corneal edema and loss of vision. The FDA regards a 30% loss as significant. Endothelial cells naturally decline by 0.5% per year. Glaucoma by itself is a known risk factor for endothelial cell loss (ECL). ECL and corneal failure are known complications of glaucoma tube surgery. Studies have shown that current GDIs have significant ECL. Endothelial cell loss after GDI surgery is probably multifactorial and may depend on the tube position in the anterior chamber (AC) and its proximity to the posterior corneal surface, the biocompatibility of the material used, chronic inflammation, tube stability and micromovements transferred from the scleral plate to the intracameral portion of the tube as well as fluid turbulence induced by the tube near the cornea. Thus, there remains a need in the art for an eye drainage device with a decreased risk of ECL.

Some known devices teach away from the direct attachment of a scleral plate due to the tendency for micro movements of the plate to cause mechanical attrition of corneal endothelial cells, erosion of the conjunctiva, and implant extrusion. However, scleral plates are important in GDIs in order to maintain a subconjunctival filtration area. Thus, there remains a need in the art for a GDI having a scleral plate which causes minimal endothelial cell loss or other post-operative complications.

### SUMMARY

As with other glaucoma surgeries, the use of GDIs carries certain risks and complications. These include early hypotony, early hypertensive phase, and long term ECD loss. Different tube designs and surgical techniques have been attempted to limit such complications with limited success. Although it is not possible to completely eliminate all these complications, it is possible to reduce their incidence. Eye drainage devices of the instant disclosure can address the whole set of complications by controlling and actively managing the aqueous flow through the tube, as well as by minimizing the interaction of the tube with the corneal endothelium.

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to provide an eye drainage device having an adjustable or extensible tube length to provide flexibility and significantly reduce the need and the length of the tube in the anterior chamber, thus reducing incidence of endothelial cell loss. In a non-limiting example, the extensible tube may comprise a proximal (attached to the scleral plate) and distal segment and a portion of the proximal segment may be corrugated to allow for longitudinal movement of the tube. In a further non-limiting example, the extensible tube may comprise a proximal tube which slidably receives a distal tube. The proximal and distal tubes can freely slide, allowing for longitudinal movement of the tube length.

It is a further object, feature, and/or advantage of the present disclosure to provide an eye drainage device having a scleral plate comprising a groove and/or locking mechanism to prevent extrusion of the extensible tube from the scleral plate, thereby stabilizing the device and minimizing risk of postoperative complications.

It is still yet a further object, feature, and/or advantage of the present disclosure to limit micromovements of the extensible tube to reduce endothelial cell loss and protect against tube erosion and exposure.

It is still yet a further object, feature, and/or advantage of the present disclosure to provide an extensible tube having a terminal open end configured to prevent extrusion of the terminal open end from an intraocular chamber of an eye following placement. In a non-limiting example, the terminal open end may be a hook shape. In a further non-limiting example, the terminal open end may be arrowhead or collar-button shaped. In a further non-limiting example, the terminal open end may be an ellipsoid shape comprising horizontal flanges.

It is still yet a further object, feature, and/or advantage of the present disclosure to manage early aqueous flow. In a non-limiting example, this may be through providing an extensible tube having optimized lumen size, more than one lumen, and/or occluding the lumen(s) of the extensible tube by temporary occlusion to restrict flow of an intraocular fluid.

It is still yet a further object, feature, and/or advantage of the present disclosure to provide a method of reducing intraocular pressure in an eye of a patient.

It is still yet a further object, feature, and/or advantage of the present disclosure to provide a method of draining fluid from an intraocular chamber in an eye of a patient.

It is still yet a further object, feature, and/or advantage of the present disclosure to provide a method of treating glaucoma in an eye of a human or animal patient.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1A shows a side perspective view of an exemplary eye drainage device according to some aspects of the present disclosure.

FIG. 1B shows a top perspective view of the eye drainage device of FIG. 1A.

FIG. 1C shows a front perspective view of an exemplary terminal open end of the eye drainage device of FIG. 1A.

FIG. 2A shows a cross-sectional, side perspective view of an exemplary terminal open end that can be contained within an eye drainage device according to some aspects of the present disclosure.

FIG. 2B shows a front perspective view of the terminal open end of FIG. 2A.

FIG. 2C shows a top perspective view of the terminal open end of FIG. 2A.

FIG. 2D shows a top perspective view of an alternative embodiment of the terminal open end of FIG. 2A.

FIG. 2E shows a top perspective view of a terminal open end (arrowhead) design.

FIG. 2F shows a front perspective view of the terminal open end of FIG. 2E.

FIG. 2G shows a top view of a terminal open end (collar-button) design.

FIG. 2H shows a front perspective view of the terminal open end of FIG. 2G.

FIG. 3 shows a bottom perspective view of an exemplary terminal open end comprising a microporous surface according to some aspects of the present disclosure.

FIG. 4 shows a schematic view of the interaction between an eye and an exemplary eye drainage device according to some aspects of the present disclosure.

FIG. 5 shows a cross-sectional, side perspective view of an exemplary junction between a proximal tube and distal tube that can be contained within an eye drainage device according to some aspects of the present disclosure.

FIG. 6 shows a schematic view of the interaction between an eye and an exemplary eye drainage device according to some aspects of the present disclosure.

FIG. 7 shows a top perspective view of an exemplary eye drainage device according to some aspects of the present disclosure.

FIG. 8 shows a front elevation, component view of an exemplary terminal open end that can be contained within an eye drainage device according to some aspects of the present disclosure.

FIG. 9A shows a top perspective, component view of an exemplary scleral plate comprising a membrane over the scleral groove that can be contained within an eye drainage device according to some aspects of the present disclosure.

FIG. 9B shows a top perspective, component view of an exemplary scleral plate comprising a membrane over the scleral groove that can be contained within an eye drainage device according to some aspects of the present disclosure.

FIG. 9C shows a cross sectional, front perspective, component view of an exemplary scleral plate comprising a membrane over the scleral groove that can be contained within an eye drainage device according to some aspects of the present disclosure.

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

Eye drainage devices of the present disclosure may have an adjustable or extensible tube length 101. Having an extensible tube length will significantly reduce the need and the length of the tube within the anterior chamber (AC) 402, thus reducing the tube's interaction with the corneal endothelium and reducing endothelial cell loss (ECL). The extensible tube 101 connecting the scleral plate 102 to the AC 402 can have a total length of about 8 mm to about 14 mm, about 8 mm to about 10 mm, or any range therein. The extensible tube 101 can be flexible or rigid, depending on desired end use. In some embodiments, the extensible tube 101 is flexible. The extensible tube 101 can be made of any suitable, biocompatible material. “Biocompatible” as used herein means inert to tissues and liquids which the device or component may contact and which will not generate an inflammation or wound response in the tissue. Suitable materials may include, for example, silicon, plastics, thermoplastics, polyethylene, polypropylene, fluorocarbon polymer, or elastomers.

In some embodiments, the scleral plate 102 is about 12 mm to about 20 mm, about 14 to about 16 mm, or any range or integer therein, in length. The scleral plate 102 can be made of any suitable, biocompatible material including, for example, silicon, plastics, thermoplastics, metals, polyethylene, polypropylene, fluorocarbon polymer, stainless steel, titanium, or elastomer. The scleral plate 102 can comprise anchor holes 110 for mounting the scleral plate 102 to the sclera of a patient's eye. Mounting the scleral plate 102 to the sclera can be accomplished via suturing or other known methods in the art.

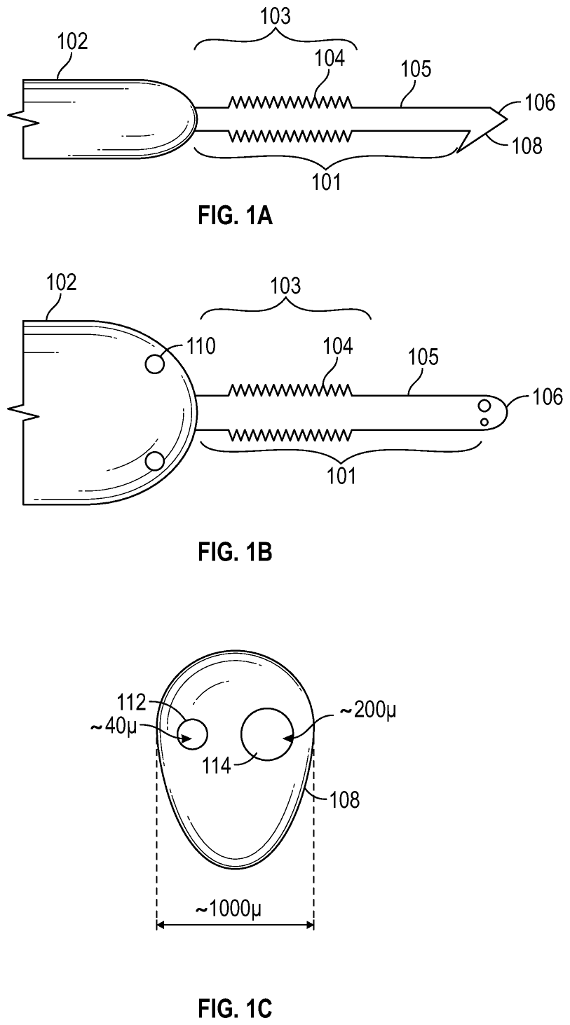

In some embodiments, the extensible tube 101 is a single tube having a proximal segment 103 and a distal segment 105, such as shown in FIGS. 1A-1B. The proximal segment 103 can be about 2 mm to about 4 mm long. The distal segment 105 can be about 4 mm to about 6 mm long. The proximal segment 103 connects to the scleral plate 102. In some embodiments the scleral plate 102 comprises a groove 602 and/or locking mechanism configured to receive at least a portion of the proximal segment 103 and prevent extrusion of the extensible tube 101 from the scleral plate 102. In some embodiments, at least a portion of the proximal segment 103 will have an “accordion/corrugated” design allowing for several millimeters of “longitudinal” flexibility (FIGS. 1A-1B). Because of this longitudinal flexibility, and after suturing the scleral plate 102 about 8 mm to about 10 mm posterior to the limbus 404, the tip of the tube (terminal open end) 106 can be inserted and anchored in the AC 402 angle without the need for trimming and without over-protrusion into the AC 402.

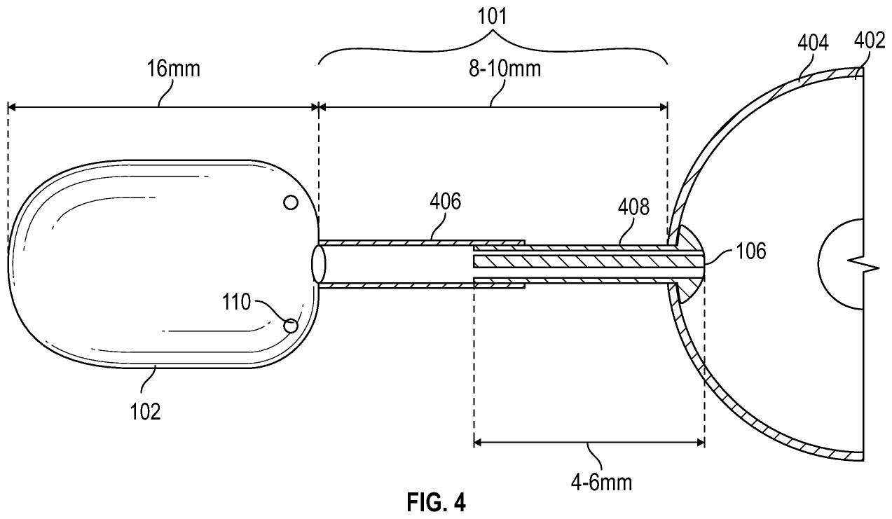

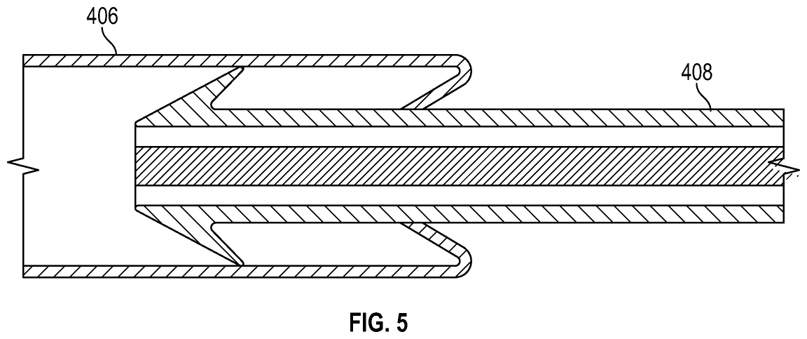

In other embodiments, the extensible tube 101 has a telescoping or sliding design and comprises two separate tubes, a proximal tube 406 and a distal tube 408 which “telescope” into each other, such as shown in FIG. 4. The distal tube 408 can have a smaller outer diameter whereas the proximal tube 406 can have a slightly larger inner diameter to accommodate the distal tube 408. In some embodiments, the proximal tube 406 is about 2 mm to about 4 mm in length. In some embodiments, the distal tube 408 will be about 4 mm to about 6 mm in length. Although the tubes should freely slide into each other, there should be enough contact and friction between their surfaces to prevent leakage around the smaller tube and force the intraocular fluid posteriorly around the plate. As shown in FIG. 5, an interlocking mechanism, such as flanges, can be designed to prevent the tubes from accidentally disconnecting during surgical manipulation and to reduce leakage around the smaller tube.

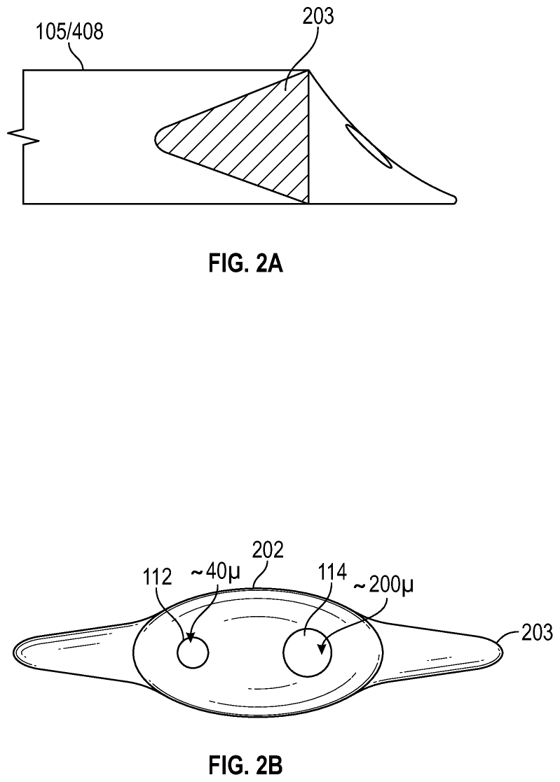

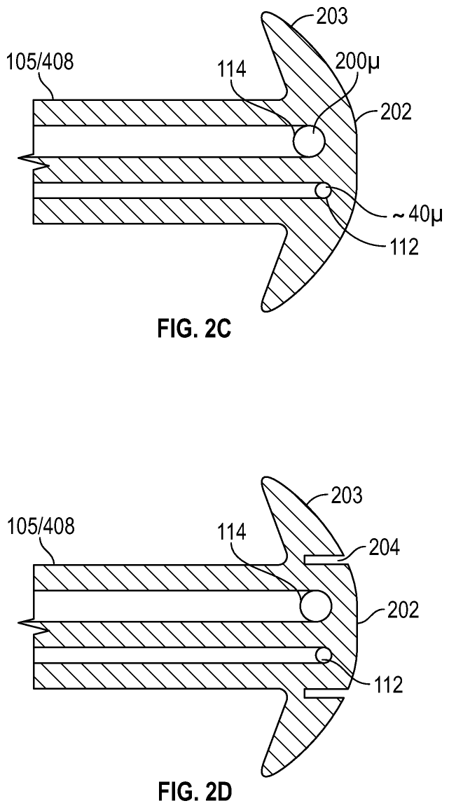

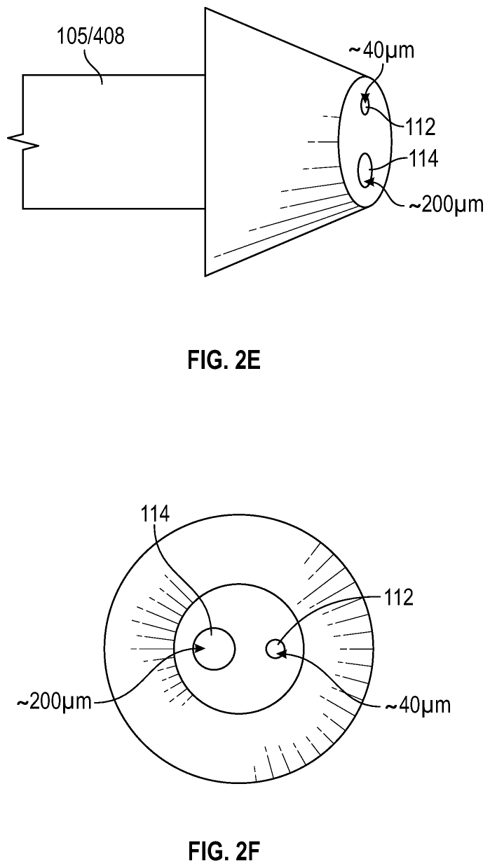

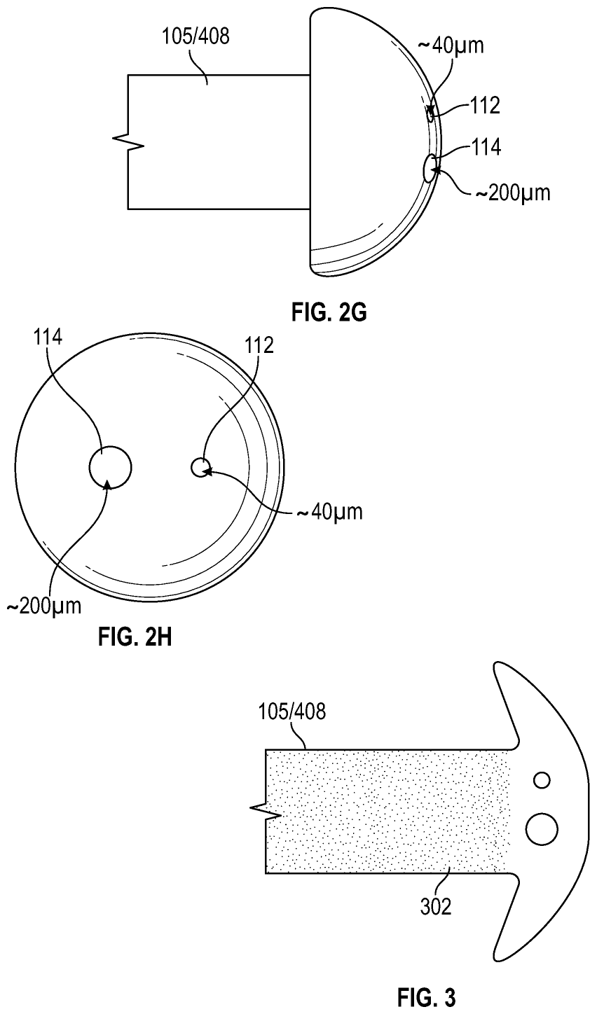

In some embodiments, the distal segment of extensible tube 105 or distal tube 408 comprises a terminal open end 106 adapted to be placed in an intraocular chamber of an eye and configured to prevent extrusion of the terminal open end 106 from the intraocular chamber following placement. One of the complications of GDIs is tube retraction from the AC. The risk of extrusion is increased when the tube is cut short into the AC or when the eye grows in size such as in pediatric patients. Embodiments of the present disclosure feature a very short length of tube in the AC to minimize contact and interaction with endothelial cells. Thus, the distal segment of extensible tube 105 or distal tube 408 comprise an anchoring mechanism to secure the tube in the AC angle. In some embodiments, the terminal open end 106 is beveled (see FIGS. 1A and 2A), has an arrowhead shape (see FIGS. 2E and 2F) or has a collar-button shape (see FIGS. 2G and 2H).

Although an extraocular stabilizing flange has been proposed in the prior art to anchor the tube at the limbus, such a design can lead to significant complications. The presence of such a piece of plastic at the limbus will increase the risks of its exposure and extrusion of the tube which can lead to more serious complications such as aqueous leak and endophthalmitis. Also, such a “bulk” near the limbus can increase the risks of corneal complications such as dellen formation. Accordingly, the present inventor has designed an internal fixation design for the terminal open end 106 of the distal segment of extensible tube 105 or distal tube 408.

In some embodiments, the terminal open end 106 of the distal segment of extensible tube 105 or distal tube 408 is a hook shape 108, as shown in FIGS. 1A-1C. The hooked terminal open end 108 can be any suitable size. In some embodiments, the hooked terminal open end has a width of, for example, from about 800 μm to about 1200 μm, from about 900 μm to about 1100 μm, from about 950 μm to about 1000 μm, or any range or integer therein.

In other embodiments, the terminal open end 106 of the distal segment of extensible tube 105 or distal tube 408 is an ellipsoid shape 202 comprising two horizontal flanges 203 on opposite sides of the ellipsoid terminal open end 202, as shown in FIGS. 2A-2D and 8. The ellipsoid terminal open end 202 can be any suitable size. In some embodiments, the ellipsoid terminal open end 202 has a length of, for example, from about 1000 μm to about 1400 μm, from about 900 μm to about 1300 μm, from about 1000 μm to about 1200 μm, from about 1100 μm to about 1200 μm, or any range or integer therein. In some embodiments, the ellipsoid terminal open end 202 has a width of, for example, from about 300 μm to about 700 μm, from about 400 μm to about 600 μm, from about 450 μm to about 550 μm, from about 520 μm to about 550 μm, or any range or integer therein (an embodiment is illustrated in FIG. 8). The horizontal flanges 203 can be any size suitable to prevent extrusion of the terminal open end from the intraocular chamber following placement. In some embodiments, the horizontal flanges 203 are about 100 μm to about 300 μm, about 150 μm to about 250 μm, about 200 μm, or any range or integer therein, in width. FIG. 8 shows an exemplary ellipsoid terminal open end 202 with exemplary dimensions.

Terminal open ends of the present disclosure are not limited to hook or ellipsoid shapes; any shape which prevents extrusion of the terminal open end from the intraocular chamber are embodied by the present disclosure. For example, the terminal open end may comprise an arrowhead (FIGS. 2E and 2F) or collar-button designs (FIGS. 2G and 2H) with a circular flange around a circular open end 106 shape, or other similar suitable designs. In some embodiments, the terminal open end has a collar-button shape with a defacto posterior circular flange that prevents extrusion and leaks around the terminal tip of the tube.

When inserted in the AC, the unique terminal open end design coupled with the longitudinal flexibility of the tube, will secure the terminal open end in the angle of the AC and prevent the retraction of the terminal open end. The length of tubing within the AC can thus be reduced to a minimum. In some embodiments, the length of tubing within the AC is less than about 1 mm, less than about 0.75 mm, less about than 0.5 mm, less than about 0.4 mm, less than about 0.3 mm, less than about 0.2 mm, less than about 0.1 mm, or any range therein. In some embodiments, the terminal open end is flush with the AC angle tissues. Beneficially, having a minimal length of tubing within the AC reduces the interaction of the device with the iris and/or endothelium. In some embodiments, a peripheral iridotomy might be needed to prevent occlusion of the terminal open end by the iris.

In embodiments having an ellipsoid terminal open end with horizontal flanges, the terminal open end can be introduced through a paracentesis performed using a microvitreoretinal (MVR) blade (19-23 G/1.4 mm-0.8 mm). In order to facilitate the introduction of the terminal open end 202 through the slit of the MVR blade, in some embodiments, the terminal open end 202 comprises flange grooves 204 separating each flange 203 from the tip of the terminal open end, as shown in FIG. 2D. As the terminal open end is passed through the slit, the flanges 203 can fold posteriorly and lay parallel to the tube. Once in the anterior chamber, the flanges 203 can deploy horizontally in the angle. Besides securing the tube in the AC angle and preventing its retraction, such a design would significantly reduce leakage around the tube which is another source of early hypotony. A similar grooved design can be employed in terminal open ends having other shapes, such as the hook design or circular design with circular flanges.

In some embodiments, the terminal open end entering the AC can have an about 90 degree bend so that aqueous flow/turbulence is directed away from the cornea and instead towards the AC angle.

In some embodiments, the terminal open end comprises a small anterior chamber protrusion 801 that protrudes slightly in the anterior chamber to prevent the iris from blocking the tube, as shown in FIG. 8.

It is to note that any of the extensible features and/or anchoring designs described above could be used for other glaucoma drainage applications such as suprachoroidal stents or any stents/tubes connecting the anterior chamber 402 to any periocular compartment or space.

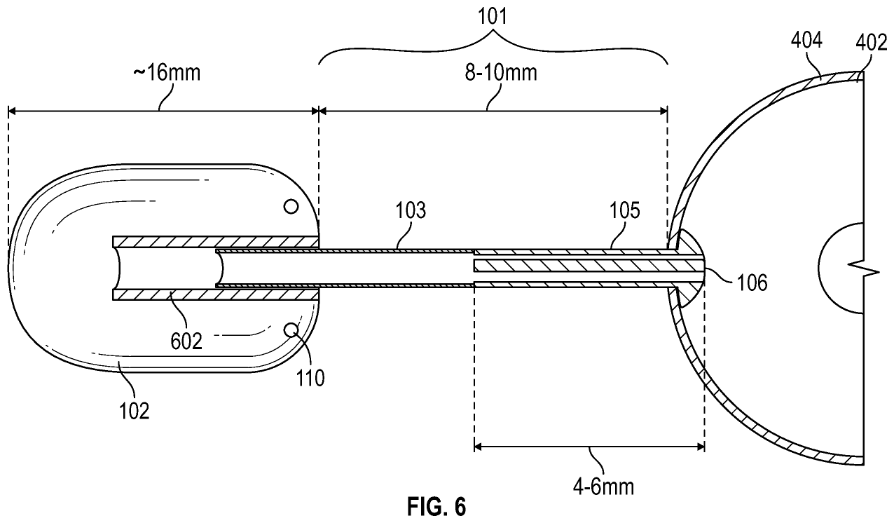

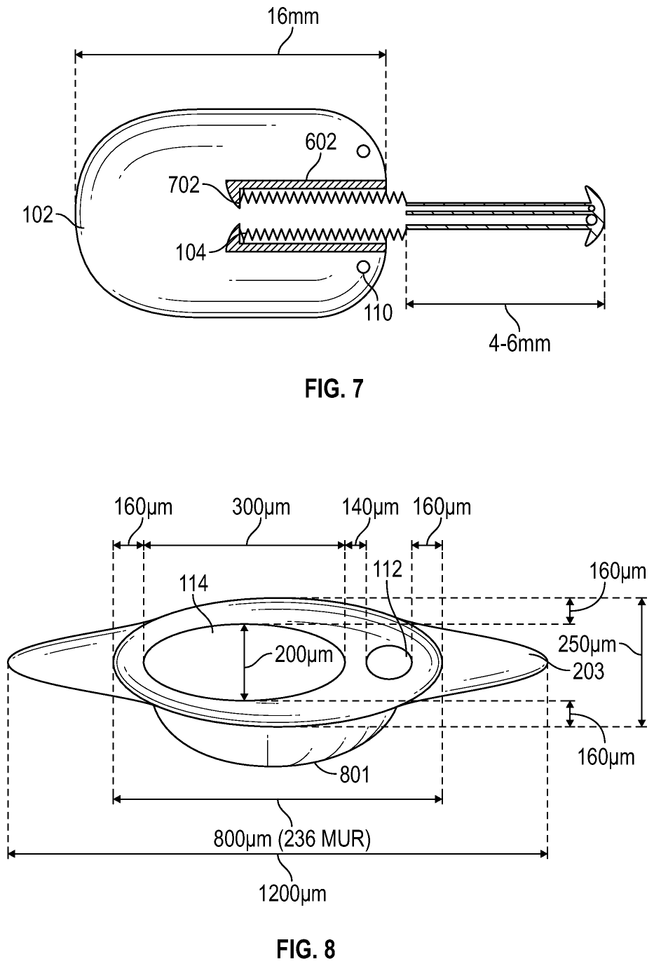

In some embodiments, the scleral plate 102 comprises a groove 602 that is configured to slidably receive the proximal segment 103 or proximal tube 406, as depicted, for example, in FIG. 6. In such embodiments, the proximal portion of the extensible tube 101 “slides” into the scleral groove 602 and can be adjusted to the desired length by sliding the extensible tube 101 into the scleral groove 602 the desired amount. In such embodiments, the extensible tube 101 and scleral plate 102 can be assembled before surgery, or the extensible tube 101 can be introduced into the scleral groove 602 of the scleral plate 102 after the scleral plate 102 has been secured to the sclera. The scleral groove 602 can have a locking mechanism 702 which anchors the extensible tube 101 and prevents extrusion of extensible tube 101 from the scleral plate 102, such as shown in FIG. 7. In some embodiments, the locking mechanism is an interlocking mechanism, similar to the interlocking mechanism used in some embodiments to connect the proximal tube 406 and distal tube 408 (see FIG. 5).

To prevent leaks, in some embodiments, the proximal segment 103 or proximal tube 406 can comprise a corrugated portion 104 completely housed within the scleral groove 602 in the body of the scleral plate 102, as shown in FIG. 7. Such a design would be preferable in case more longitudinal flexibility is needed and/or in case there is too much leakage around the tubes in the telescoping/sliding designs.

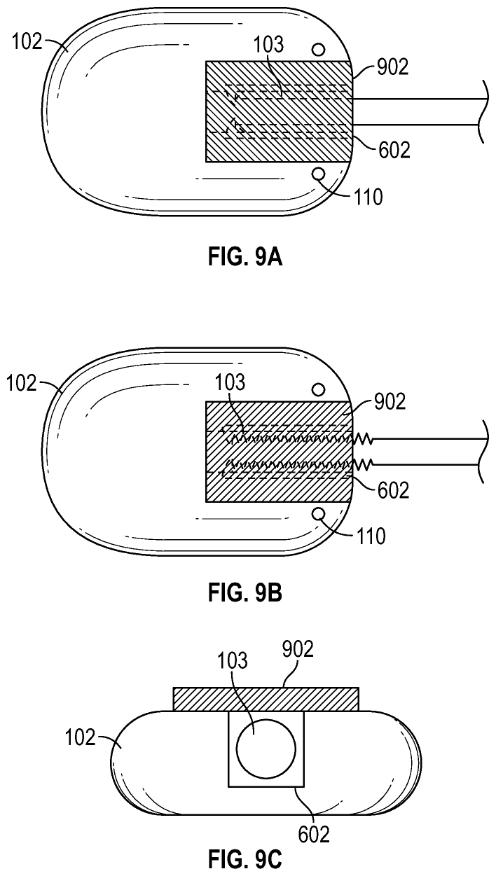

In embodiments comprising a scleral groove 602 in the scleral plate 102, there may be micromovement of the extensible tube 101 in the scleral groove 602 due to ocular motility and/or due to ocular pulsations. These macro/micromovements can increase the risk of scarring by stimulating low-level activation of the wound healing response and increased collagen scar formation. To reduce scarring, in some embodiments, the scleral groove 602 housing the extensible tube 101 in the scleral plate 102 can be covered by a thin membrane 902 made of the same material as the plate itself (FIGS. 9A-9C). Without being limited by theory, the membrane 902 shields the micromovements of the extensible tube 101 in the scleral groove 602 from the surrounding orbital tissues and from the healing/scarring processes around the scleral plate 102. The micromovements involving parts of the tube that are external to the scleral plate 102 would be less consequential to the success of the GDI, particularly because the extensible tube 101 between the scleral plate 102 and the corneal limbus 404 is typically covered by a patch graft.

Beneficially, embodiments of the present disclosure not only allow for longitudinal flexibility of the tube, but also can act as a damper in the transmission of micromovements from the scleral plate 102 to the anterior chamber 402 through the extensible tube 101. Without being limited by theory, it is believed dampening micromovements is protective against endothelial cell attrition and can reduce the incidence of ECL. To further add to this dampening effect, some or all of the distal segment of extensible tube 105 or distal tube 408 can have a microporous outer surface 302, as shown in FIG. 3. A thin layer of porous silicone material with micropores of at least about 20 μm in diameter can be wrapped around and attached to the tube using medical grade silicone adhesive. This will allow soft tissue infiltration and “fixation” of the distal tube to the surrounding ocular tissues, thus limiting its micromovements. Otherwise, the distal tube, or part of it, can be fabricated with a microporous outer surface. The microporous outer surface around that section of the extensible tube can further prevent micromovements. Besides protecting the corneal endothelial cells, limiting micromovements of the tube can be protective against tube erosion and exposure near the limbus. Tube exposure occurs in 2-5% of patients who receive GDIs and has been reported as high as 30.8%. It can be a cause of failure or catastrophic complications of GDIs such as endophthalmitis. Although multifactorial in origin, micromovements are cited among the causes of tube exposure. Thus, embodiments of the present disclosure which have a dampening effect on micromovements can decrease tube exposure and decrease the risk of GDI complications and failure.

### Managing Early Aqueous Flow and Early Hypotony

Extensible tubes 101 of the present disclosure have at least one lumen. In some embodiments, the extensible tube 101 has a single lumen across the entire length. In some embodiments, the extensible tube 101 has two lumens (see FIGS. 1C, 2B-2D, 4, 5, 6). In embodiments with two lumens, the extensible tube 101 may have two lumens across the entire length or two lumens only at the distal segment 105 or distal tube 408. The lumens may be the same size or different sizes. In some embodiments, the extensible tube 101 comprises a small lumen 112 and a large lumen 114, as shown in FIGS. 1C and 2B. The small lumen 112 can have an interior diameter of between about 35 μm to about 55 μm, about 35 μm to about 50 μm, about 40 μm to about 50 μm, or any range or integer therein. The large lumen 114 can have an interior diameter of between about 100 μm to about 300 μm, about 150 μm to about 250 μm, about 150 μm to about 200 μm, or any range or integer therein.

Normal aqueous flow in adults is variable and can fluctuate between 2.75+/−0.63 μl/min during waking hours and 1.4+/−0.19 μl/min during sleep. Preventing early aqueous flow into the subconjunctival space can prevent early hypotony as well as reduce the hypertensive phase ultimately leading to thinner, more permeable capsule. Such a thin capsule can also lead to a higher long term GDI success rate. In order to prevent early aqueous flow, the extensible tube 101 can be occluded with a temporary occlusive, such as a biodegradable membrane or plug that spontaneously dissolves a few weeks after insertion. In embodiments having two lumens as described above, the smaller lumen 112 can have a temporary occlusive that dissolves after about 2 to about 3 weeks from insertion while the larger lumen 114 can have a temporary occlusive that dissolves after about 6 to about 8 weeks from insertion by using a different temporary occlusive or simply a larger/longer occlusive of the same material as used in the smaller lumen. Alternatively, the lumen(s) can be occluded with a thin membrane that can be opened after surgery using a Nd YAG laser. Biodegradable suture can also be used to ligate the lumen(s) externally. A combination of the above techniques can also be used.

In order to prevent extreme hypotony, particularly in the early postoperative phase after the lumen occlusion has been reversed, in some embodiments the distal segment 105 or distal tube 408 (4-6 mm long) can have an interior diameter of between about 35 μm to about 55 μm, about 35 μm to about 50 μm, about 40 μm to about 50 μm, or any range or integer therein. According to Poiseulle's equation, this will lead to a pressure gradient of ˜5 mmHg across the distal segment at an aqueous flow rate of 1.4 μl/min. In theory, this means that the IOP will not go below ˜5 mmHg even in the absence of any capsular resistance to flow and extreme hypotony can be avoided in the early post operative period. However, such a design may result in the formation of resistance in series (tube and capsule) with the possibility of higher final IOP after a fibrotic capsule has formed around the scleral plate.

To avoid such a problem, in some embodiments, the extensible tube 101 can comprise a small lumen 112 and a large lumen 114 as described above. The small lumen 112 will be functional starting around 2-6 weeks after surgery (after dissolution or reversal of the temporary occlusive), while the large lumen 114 would have no or minimal resistance to flow. The terminal open end of this larger lumen may be occluded by a temporary occlusive, such as a thin membrane that can be opened using a Nd YAG laser once the IOP reaches about 8 to about 10 mmHg, a plug or membrane that spontaneously dissolves after 6-8 weeks, or a biodegradable suture. This approach can lead to more biodegradation in the capsule and better long term IOP control.

Alternatively, in other embodiments, an extensible tube 101 with a single large lumen can be designed. The terminal open end can be occluded by a temporary occlusive, such as a biodegradable flow restricting membrane that dissolves over several months after implantation, or by a semipermeable membrane that initially provides high resistance to flow preventing early hypotony. Once the risk of hypotony has been minimized, the semipermeable membrane can be perforated using a laser according to known techniques.

From the foregoing, it can be seen that the present invention accomplishes at least all of the stated objectives.

### LIST OF REFERENCE CHARACTERS

The following table of reference characters and descriptors are not exhaustive, nor limiting, and include reasonable equivalents. If possible, elements identified by a reference character below and/or those elements which are near ubiquitous within the art can replace or supplement any element identified by another reference character.

TABLE 1
List of Reference Characters

| | |
|---|---|
| 101 | Extensible tube |
| 102 | Scleral plate |
| 103 | Proximal segment of extensible tube |
| 104 | Corrugated portion of the proximal segment |
| 105 | Distal segment of extensible tube |
| 106 | Terminal open end |
| 108 | Hooked terminal open end |
| 110 | Scleral plate holes |
| 112 | Small lumen |
| 114 | Large lumen |
| 202 | Ellipsoid terminal open end |
| 203 | Horizontal flange |
| 204 | Flange groove |
| 302 | Microporous surface of distal segment or distal tube |
| 402 | Anterior chamber of eye |
| 404 | Limbus of eye |
| 406 | Proximal tube |
| 408 | Distal tube |
| 602 | Scleral plate groove |
| 702 | Locking mechanism for proximal tube |
| 801 | Anterior chamber protrusion |
| 902 | Membrane |

### Glossary

Unless defined otherwise, all technical and scientific terms used above have the same meaning as commonly understood by one of ordinary skill in the art to which embodiments of the present invention pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

What is claimed is:

1. An implantable eye drainage device, comprising:
a scleral plate comprising a groove and/or locking mechanism;
an extensible tube having at least one lumen, wherein the extensible tube comprises a proximal segment and a distal segment,
wherein the groove and/or locking mechanism of the scleral plate is configured to receive at least a portion of the proximal segment of the extensible tube and prevent extrusion of the extensible tube from the scleral plate,
wherein the distal segment of the extensible tube comprises a terminal open end adapted to be placed in an intraocular chamber of an eye and configured to prevent extrusion of the terminal open end from the intraocular chamber following placement.

2. The implantable eye drainage device of claim 1, wherein at least a portion of the proximal segment of the extensible tube is corrugated.

3. The implantable eye drainage device of claim 1, wherein the extensible tube is from about 8 mm to about 14 mm in length.

4. The implantable eye drainage device of claim 1, wherein the scleral plate comprises a groove and wherein the groove slidably receives at least a portion of the proximal segment of the extensible tube.

5. The implantable eye drainage device of claim 4, wherein the portion of the proximal segment of the extensible tube slidably received by the groove is corrugated.

6. The implantable eye drainage device of claim 1, wherein the terminal open end of the distal segment of the extensible tube is a hook shape.

7. The implantable eye drainage device of claim 1, wherein the terminal open end of the distal segment of the extensible tube is an ellipsoid shape comprising two horizontal flanges, an arrowhead shape, or a collar-button shape comprising a posterior circular flange.

8. The implantable eye drainage device of claim 1, wherein the extensible tube comprises two lumens.

9. An implantable eye drainage device, comprising:
a scleral plate comprising a groove and/or locking mechanism;
a proximal tube comprising a first end and a second end;
a distal tube comprising a first end and a terminal open end, wherein at least a portion of the first end of the distal tube is slidably received by at least a portion of the second end of the proximal tube;
wherein the groove and/or locking mechanism of the scleral plate is configured to receive at least a portion of the first end of the proximal tube and prevent extrusion of the proximal tube from the scleral plate,
wherein the terminal open end of the distal tube is adapted to be placed in an intraocular chamber of an eye and configured to prevent extrusion of the terminal open end from the intraocular chamber following placement.

10. The implantable eye drainage device of claim 9, wherein there is sufficient contact between the first end of the distal tube and the second end of the proximal tube to prevent leakage of a fluid flowing therethrough.

11. The implantable eye drainage device of claim 9, wherein the proximal tube and the distal tube have a combined total length of between about 8 mm to about 14 mm.

12. The implantable eye drainage device of claim 9, wherein the scleral plate comprises a groove and wherein the groove slidably receives at least a portion of the first end of the proximal tube.

13. The implantable eye drainage device of claim 12, wherein the portion of the first end of the proximal tube slidably received by the groove is corrugated.

14. The implantable eye drainage device of claim 1, wherein the terminal open end of the distal tube is a hook shape.

15. The implantable eye drainage device of claim 1, wherein the terminal open end of the distal tube is an ellipsoid shape comprising two horizontal flanges.

16. A method of treating glaucoma in an eye of a human or animal patient, comprising:
mounting a scleral plate to the sclera of the eye, wherein the scleral plate comprises a groove and/or locking mechanism configured to receive at least a portion of a first end of a proximal tube and prevent extrusion of the proximal tube from the scleral plate, wherein at least a portion of a second end of the proximal tube slidably receives at least a portion of a first end of a distal tube;
inserting a terminal open end of the distal tube in an intraocular chamber of the eye, wherein the terminal open end is configured to prevent extrusion of the terminal open end from the intraocular chamber following placement.

17. The method of claim 16, wherein the distal tube comprises a first lumen and a second lumen, wherein the first lumen has an interior diameter of between about 35 μm to about 55 μm and the second lumen has an interior diameter of between about 100 μm to about 300 μm.

18. The method of claim 17, wherein the first and/or the second lumen is occluded by temporary occlusive to restrict flow of an intraocular fluid.

19. The method of claim 18, wherein the temporary occlusive is a biodegradable suture and/or a biodegradable membrane.

20. The method of claim 15, wherein the terminal open end of the distal tube is an arrowhead shape, a collar-button shape, a hook shape or an ellipsoid shape comprising two horizontal flanges.
